(19) United States
(12) Patent Application Publication
(10) Pub. No.: US 2008/0014248 A1
(43) Pub. Date: Jan. 17, 2008

(54) Photosensitizer containing indole-3-alkylcarboxylic acid, and kit for photodynamic therapy containing the same

(76) Inventors: Kyoung-Chan Park, Seoul (KR); Dong-Seok Kim, Seoul (KR); So-Young Kim, Seoul (KR)

Correspondence Address: THE NATH LAW GROUP, 112 South West Street, Alexandria, VA, 22314, US

(21) Appl. No.: 11/822,431

(22) Filed: Jul. 5, 2007

(30) Foreign Application Priority Data
Jul. 7, 2006 (KR) 10-2006-0063841

(51) Int. Cl. A61K 31/404; A61K 9/02; A61K 9/12; A61K 9/20; A61K 9/48; A61N 5/06; A61N 5/067; A61P 35/00

(52) U.S. Cl. 424/436; 424/45; 424/451; 424/464; 514/419; 607/88; 607/89; 607/94

(57) ABSTRACT

The present invention relates to a photosensitizer containing indole-3-alkylcarboxyl acid (ICA), and kit for photodynamic therapy containing the same. More specifically, the present invention is directed to a pharmaceutical composition comprising ICA or a pharmaceutically acceptable salt thereof, and a novel method for photodynamic therapy using ICA as a photosensitizer.

### TECHNICAL FIELD

The present invention relates to a photosensitizer containing indole-3-alkylcarboxyl acid (ICA), and kit for photodynamic therapy containing the same. More specifically, the present invention is directed to a pharmaceutical composition comprising ICA or a pharmaceutically acceptable salt thereof, and a novel method for photodynamic therapy using ICA as a photosensitizer.

### BACKGROUND ART

Photodynamic therapy (PDT) is one of the new promising therapies for the treatment of cancer. It involves three key components: a photosensitizer, light, and tissue oxygen. It is also being investigated for treatment of psoriasis and acne. A photosensitizer is a chemical compound that can be excited by light of a specific wavelength. This excitation uses visible or near-infrared light. When the photosensitizer and an oxygen molecule are in proximity, an energy transfer can take place that allows the photosensitizer to relax to its ground singlet state, and create an excited singlet state oxygen molecule. Singlet oxygen is a very aggressive chemical species and will very rapidly react with any nearby biomolecules. Ultimately, these destructive reactions will result in cell killing through apoptosis or necrosis.

Specifically, ICA is stimulated by ultraviolet light or visible light. Among visible light, green and blue light is preferred. When ICA is stimulated with light, ICA can emit free radical and destroy cancer cells or unnecessary tissue or bacteria etc.

PDT uses laser, or other light sources, combined with a light-sensitive drug (sometimes called a photosensitising agent) to destroy cancer cells. A photosensitizing agent is a drug that makes cells more sensitive to light. Once in the body, the drug is attracted to cancer cells. It is inactive until exposed to a particular type of light. When the light is directed at the area of the cancer, the drug is activated and the cancer cells are destroyed. Some healthy, normal cells in the body will also be affected by PDT, although these cells will usually heal after the treatment. PDT may be used to treat cancers of the skin, or those that are on, or near, the lining of internal organs, such as cancers of the head and neck area, the lining of the mouth, the lining of the lung, the lining of the esophagus, the lining of the stomach, the lining of the bladder, the lining of the bile ducts. Furthermore, PDT can applied for the treatment of benign disease such as psoriasis or acne. Still the PDT has many limitations. Thus, safe and effective photodynamic therapy method or photodynamic therapy kits need to be developed.

In order to understand the role of photodynamic therapy (PDT) in the treatment of malignant tumors, one needs to consider state-of-the-art routine approaches to this problem. All therapies can be classified into 1) local (in which the primary tumor is treated) and 2) systemic (in which disseminated cancer is treated).

The main types of local therapy include surgical treatment and radiotherapy. Local treatments are generally aimed at the destruction of the primary tumor and metastases in regional lymphatic nodes. In many cancer patients, these therapeutic methods are efficient by themselves. Systemic treatment usually means chemotherapy or some kind of immunotherapy. Systemic approach is employed to treat distant macro- and micro-metastases. It is directed mainly at the survival prolongation and surgical treatment improvement. Besides that, systemic treatment removes local tumor manifestations. Photodynamic therapy is a local therapy, aimed at the treatment of local tumor manifestations. However, it can be stated that PDT will not be applied in the treatment of all forms of cancer because of superficial effects of PDT. However, it is reported that photosensitizers are selectively accumulated in tumor cells, as compared to normal tissues (Gomer and Dogherty, 1979; Jori, 1996; Young, et al., 1996; Dougherty, et al., 1998). Potentially, PDT specificity can be achieved by photosensitizer accumulation and by exposed area confinement. This will cause serious damage to tumor cells and an insignificant damage of healthy tissues. Such an enhancement of the therapeutic effect gives PDT salient advantages over other therapeutic techniques.

Photodynamic therapy research began in 1980, and by 1990 PDT was approved for clinical surgery operations in Canada, Germany, and Japan. The first PDT application, which was approved by the FDA in the United States, was the palliative treatment of obstructive esophagus cancer. Then, in September 1997 FDA approved the first treatment of lung cancer using PDT.

However, the presently operated PDT is restricted because the light is unable to penetrate when treating large tumors, and in addition to the high cost of the existing porphyrin photosensitizer there are risks for side effects. Thus, due to the low consistency when treating tumors the effectiveness of the existing treatment is questionable.

New generation photosensitizing agents such as porphyrins, chlorines, bacteriochlorins, porphycenes, etc. are being researched extensively (J Org. Chem., 63, 1998, 1646-1656). Among these agents, much research continues to be carried out on pheophytins, which is chlorophyll with its metal ions removed. Pheophytins not only absorb light with long wavelengths better than Photofrin™, a derivative of hematoporphyn, but can also be separated and prepared with high purity. However, despite extensive research, no real substantial results have been attained yet. Consequently, there is great demand for the development of an effective photosensitizer for use in PDT.

### TECHNICAL SOLUTION

In order to overcome the drawbacks of the conventional photosensitizer, the present inventors had studied for a long time to find a novel photosensitizer, and finally provide a novel photosensitizer with high cancer system selectivity and minimal side effect occurrence for use in photodynamic therapy; a pharmaceutical composition for the photosensitzer to be used in photodynamic therapy; a method for administering the pharmaceutical composition; and a kit for photodynamic therapy containing the photosensitizer.

### DISCLOSURE OF INVENTION

The primary object of the present invention is to provide a photosensitizer for treatment or prevention of cancer, which comprises a compound of formula (I) or a pharmaceutically acceptable salt thereof: 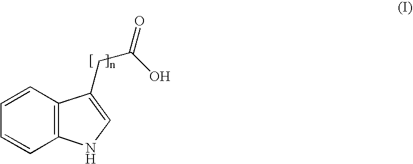
wherein n is an integer of 0 to 3.

Another object of the present invention is to provide a pharmaceutical composition which comprises a compound of formula (I) or a pharmaceutically acceptable salt thereof, and a pharmaceutically acceptable carrier or diluent, wherein n is an integer of 0 to 3.

Yet another object of the present invention is to provide a method for administrating the pharmaceutical composition which comprises a compound of formula (I) or a pharmaceutically acceptable salt thereof, and a pharmaceutically acceptable carrier or diluent, wherein the administering route is selected from the group consisting of topical application, intravenous injection, intramuscular injection, intra-cranial injection, intra-tumoral injection, intraepithelial injection, trans-epidermal injection, esophageal administration, intra-peritoneal administration, intra-arterial injection, intra-articular injection and oral administration.

Yet another object of the present invention is to provide a photodynamic therapy kit which comprises: i) a pharmaceutical composition containing a compound of formula (I) at a concentration of 0.001 wt % to 30 wt %; and ii) a light emitting device for irradiation of light of wavelength of 280 nm to 1,000 nm.

The objective of the present invention can be achieved by providing a highly sensitive and selective photodynamic therapy with little side effects. This invention relates to derivatives of indole-3-alkylcarboxylic acid (ICA) and their use as a photosensitizer in photodynamic therapy (PDT). More specifically, ICA derivatives can be photo-activated by ultraviolet light or visible light, most effectively by green and blue light. When ICA is irradiated, photo-activated ICA can destroy cancer cells or disease tissue.

Another purpose of this invention is to provide photodynamic cancer therapy kit using this combination.

Indole-3-acetic acid, also known as IAA, is a member of the group of phytohormones called auxins. IAA is generally considered to be the most important native auxin and plant growth regulator.

Indole-3-acetic acid (IAA) is the plant growth hormone, which also possesses bioactive properties on yeast and animal cells. It has been reported that IAA in association with horseradish peroxidase (HRP) leads to the death of human cancer cells, and it could be used as a novel anticancer agent (Kim D S et al. Oxidation of indole-3-acetic acid by horseradish peroxidase induces apoptosis in G361 human melanoma cells. Cell Signal 2004; 16: 81-8; Greco et al. Mechanisms of cytotoxicity induced by horseradish peroxide/indole-3-acetic acid gene therapy. J Cell Biochem 2002; 87: 221-32; Huang et al. Apoptosis of pancreatic cancer BXCP-3 cells induced by indole-3-acetic acid in combination with horseradish peroxide. World J Ganstroenterol 2005; 11: 4519-23). Furthermore, the present inventors proposed that IAA/HRP-induced free radicals lead to the apoptosis of the cells, because IAA/HRP-induced apoptosis is blocked by antioxidants. However, it is not known which kind of free radicals are involved and how they function in IAA/HRP-mediated reactions.

IAA is an interesting substance, because IAA alone is non-toxic and well tolerated in humans, but becomes active after oxidative decarboxylation by HRP. Therefore, it has been suggested that IAA can be activated only in tumors, if HRP is targeted to cancer cells. Based on these studies, three concepts for targeting HRP to tumors are suggested: antibody directed enzyme prodrug therapy (ADEPT), polymer directed enzyme prodrug therapy (PDEPT), and gene directed enzyme prodrug therapy (GDEPT) (Use of indole-3-acetic acid derivatives in medicine, U.S. Pat. No. 6,890,948). However, ADEPT can cause immunological problems because of foreign proteins. In GDEPT, the enzyme may be expressed only intra-cellularly. Thus, it is not easy to deliver enough amount of HRP for the activation of IAA in tumor tissue.

Thus, the present inventors investigated whether light could activate indole-3-alkylcarboxylic acid (ICA) and produce free radicals, which could induce necrosis of cancer cells. Indeed, it was found that ICA is potently activated by ultraviolet light and visible light. Among visible light, especially green and blue light was most effective in inducing free radicals by ICA.

Recently, it is reported that IAA enhances the efficacy of photodynamic cancer therapy by forming free radicals (Folkes, L. K. and Wardman, P. Enhancing the efficacy of photodynamic cancer therapy by radicals from plant auxin (indole-3-acetic acid). Cancer Res, 63: 776-779, 2003; Folkes, L. K. and Wardman, P. Oxidative activation of indole-3-acetic acids to cytotoxic species—a potential new role for plant auxins in cancer therapy. Biochem Pharmacol. 2001 Jan. 15; 61(2):129-36). In their report, they used phenothiazinium dye and toluidine blue dye as a photosensitizer for the oxidation of IAA. Oxidative activation of IAA by peroxidase or other photocatalysts including phenothiazinium dye or riboflavin, is toxic to cancer cells or microorganisms. (Fukuyama T T and Moyed H S. Inhibition of cell growth by photooxidation products of indole-3-acetic acid. J Biol Chem 1964, 239(7):2392-2397). But IAA is not known as a photosensitizer and has not been tried for the treatment of cancer by combination with light. In this invention, the present inventors use ICA is a photosensitizer and both ultraviolet light and visible light are effective in activation of ICA. Particularly, green and blue light was effective in activation of ICA. Compared to ultraviolet light, visible light can penetrate deeply into the tissue. Thus, visible light can be efficient in delivery of light for the activation of ICA in the tumor tissue. Furthermore, ICA with light combination was toxic only to tumor cells. Our results showed that normal human fibroblast was resistant to toxic effects of ICA and HRP or light combination (FIGS. 2, 3). Thus, the present invention can provide a method of highly sensitive and selective photodynamic therapy with little side effects.

For this purpose, a pharmaceutical photosensitizing composition comprising a derivative of compound, of formula (I): 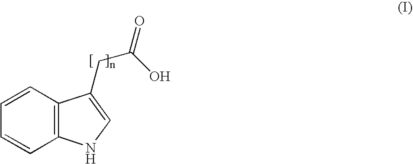
wherein n is an integer of 0 to 3.

In addition, the present invention provides the composition of indole-3-alkylcarboxylic acid (ICA) with structure of said formula 1, which comprises effective consistituents for photosensitizer quality, and the therapeutically effective amount for photodynamic therapy.

In addition, the present invention provides a photodynamic therapy kit containing photosensitizer ICA with structure of said formula 1 and (a light emitting device for in vivo or in vitro light delivery.) In this invention, ICA does not need any photocatalysts for activation by light. In addition, any wavelength can activate ICA, however, ultraviolet light (>280 nm) was found to be the most effective for the activation of ICA. Longer wavelength light can penetrate deeply into the tissue. Thus, any wavelengths between 280 through 1,000 nm light can be used effectively. However, experimental results showed that blue and green light (between 400 through 600 nm) was the most effective for the activation of ICA.

In the present invention, the light emitting device can be a light emitting diode system, laser diode, dye laser, halogen metal lamp, flash lamp, filtered fluorescent or any kinds of lamp for photodynamic therapy or any system for the delivery of light to the inside of the body through laser fiber.

Furthermore, in this invention, ICA can be either photo-activated after injection into the body or photo-activated before injection into the body. Because there is no limit for energy intensity of the emitted light during in vitro ICA activation, when light intensity is low the duration time of exposure and/or frequency of emission may be increased, and when light intensity is high the duration time of exposure and/or frequency of emission may be decreased during activation.

If the light intensity is too low there will not be sufficient penetration of the target tissue and thus effective light activation will not occur. If the light intensity is too high, on the other hand, necrosis of normal tissue may occur. Thus, the intensity of light should be maintained between 1-100 J/cm.

Further, if the pulse exposure time is too short or delivery frequency is too low then the effectiveness of light activation will be diminished, on the other hand if pulse exposure time is too long or delivery frequency is too high, necrosis of normal tissue may occur. Thus, the pulse exposure time should be maintained between 0.1-500 ms and frequency of emission should be maintained between 1-100 emissions.

The ICA composition in the PDT has ICA with photosensitizing activity that can consist of 0.001%-99% of the weight, but more desirable is 0.001%-30% of total content weight. In order to maintain sufficient ICA photosensitivity effect and therapeutic effect, ICA weight should be at least 0.001%. The composition can be used in a liquid, semi-solid, solid or aerosol state such as aqueous or nonaqueous suspensions, solutions, creams, ointments, syrups, suppositories, tablets, capsules, microdrop sprays, etc. In addition, necessary delivery vehicles can be added to the composition and similar formulations. Also, the said composition may contain preservatives, stabilizers, buffers, pH regulators, sweetening compounds, aromatic compounds, dyes, etc. for storage and administration methods. In addition, other types of drugs may be added to the composition based on the objective of the therapy.

ICA can be either photo-activated after injection into the body or photo-activated before injection into the body. In order to be photo-activated in the body, ICA should be irradiated with light after administration into the body. The photosensitizing compound, including ICA, can be delivered through one of various administration methods including topical application, intravenous injection, intra-muscular injection, intra-cranial injection, intra-tumoral injection, intraepithelial injection, trans-epidermal injection, esophageal administration, intra-peritoneal administration, intra-arterial injection, intra-articular injection, and oral administration.

A pharmaceutical combination of this invention can be applied to the treatment or prevention of conditions such as skin or skin associated diseases (actinic keratosis, warts, Bowen's disease, acne, basal cell carcinoma, squamous cell carcinoma, malignant melanoma, psoriasis, lichen planus etc), oral and gastrointestinal tract diseases (stomach cancer, duodenal cancer, gastritis etc), urinary or urinary related diseases (prostate cancer, prostatitis, cervix cancer, endometritis, uterus cancer, pelvic inflammatory disease, etc), respiratory or related diseases (lung cancer etc), circulatory or related diseases (leukemia etc), diseases related to head and neck (brain tumor, thyroid cancer, larynx cancer, laryngitis, nose cancer, rhinitis, tongue cancer etc), lymphoreticular disorders (lymphoma etc), infectious disease including micro-organism, virus, parasitic disorders (impetigo, furuncle, carbuncle etc).

### BRIEF DESCRIPTION OF DRAWINGS

FIG. 1 shows a graphical analysis from example 1 of the cytotoxic effects of indole-3-acetic acid with Horseradish Peroxidase (HRP).

FIGS. 2a, 2b and 2c show graphical analysis from example 2 of cytotoxic effects of IAA/HRP on various cell types.

FIG. 3 shows a graphical analysis from example 3 of the cytotoxic effects of IAA with UVB irradiation.

FIG. 4 shows a graphical analysis from example 4 of the degrees of photo-activation of IAA by different wavelengths of light

FIG. 5 shows an image from example 5 of treatment of cancer with Intense Pulsed Light (IPL) alone which produced no effect on the cancer cells.

FIG. 6 shows an image from example 6 of treatment of cancer by combination of IAA and IPL which shows the effects of cytotoxicity on the cancer cells.

FIG. 7 shows an image from example 7 of the prevention of cancer by combination of IAA and IPL

### BEST MODE

Hereinafter, the present invention will be described in greater detail with reference to the following examples. The examples are given only for illustration of the present invention and not to be limiting the scope of the present invention.

### EXAMPLE 1

### Cytotoxic Effects of indole-3-acetic acid with HRP

The present experiment confirmed that there are cytotoxic effects of IAA when used with HRP, but absolutely no cytotoxic effects when IAA is used alone.

(Cell Culture)

G361 human melanoma cell line (ATCC, Rockville, Md.) was cultivated in a 5% CO₂, 37□, 10% fetal bovine serum (FBS), and 50 μg/mL penicillin containing RPMI 1640 culture (WelGene, Daegu, Korea).

(G361 Cell Toxicity Experiment)

The cells cultivated in the medium were divided into 24 wells (4×10⁴/well), and was then cultivated in a medium without FBS for 24 hours. Cytotoxic effect was measured using MTT (3,4-dimethylthiazol-2-yl)2,5-diphenyltetrazolium bromide) assay (Kim, D. S., Jeon, S. E., Park, K. C. Cell Signal, 16, 81-8, 2004).

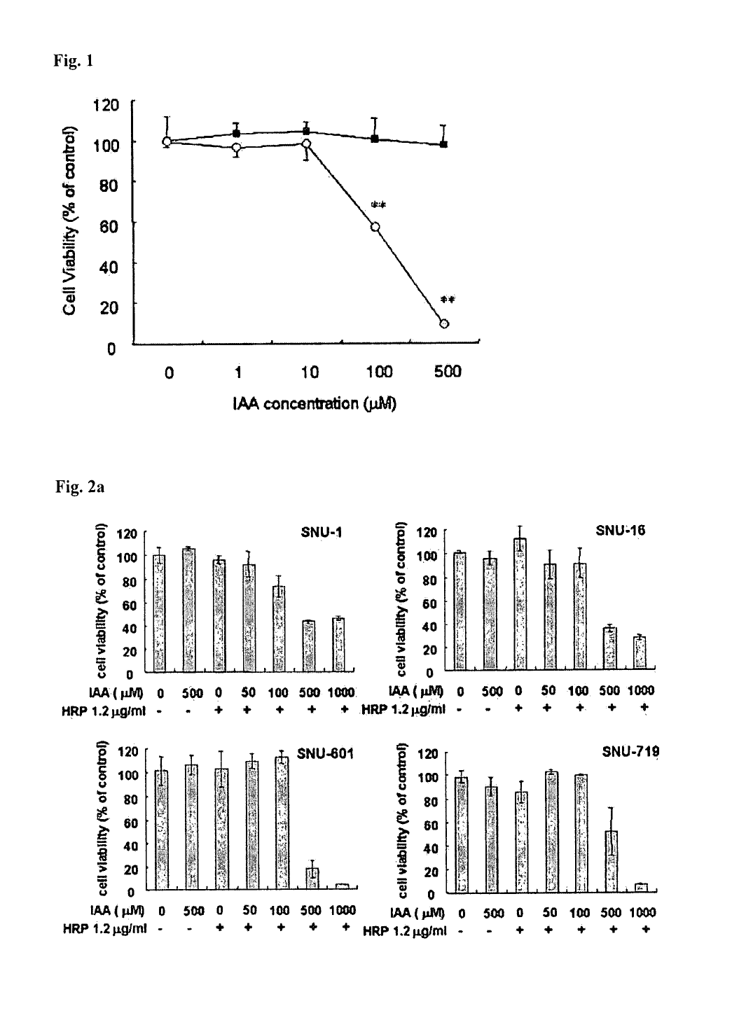

An HRP (1.2 mg/ml, Sigma, St. Louis, Mo.) treated group (indicated by the ‘∘’ in FIG. 1) and a group untreated with HRP (indicated by the ‘▪’ in FIG. 1) were separated, each with a final IAA density between 1 and 500 mM in the culture. After cultivating for 20 hours, 0.5 mg/Ml of MTT was added to the culture and cultivated for 4 more hours. After adding and dissolving 1 ml of dimethylsufoxide solution in the well, the absorption was measured using ELISA reader (TECAN, Salzburg, Austria) at 540 nm. The results for absorption and cell viability rate were calculated and are shown in FIG. 1. The viability rate for the control group was set at 100%.

(Calculations)
Cell viability(%)=(experimental group/control group absorption)×100

As shown in FIG. 1, the group with IAA alone (▪) showed no signs of cytotoxic effects, while the group with both IAA and HRP (∘), with a density of at least 100 mM, showed definite cytotoxic effects. With these results, it can be confirmed that HRP is essential for activation.

### EXAMPLE 2

### Cytotoxic Effects of IAA/HRP on Various Cell Types

After cultivating various types of tumor cells and fibroblasts, IAA/HRP treatment was administered. It was determined that the combination of IAA and HRP was toxic to most of the cancer cells but not toxic to normal human fibroblasts.

Stomach cancer cell line (SNU1, SNU16, SNU601, SNU719, Korean Cell Bank, Seoul, Korea), and lung cancer cell line (NCI-H157, NCI-H1264, Korea Cell Bank, Seoul, Korea) were cultivated in a 5% CO₂, 37□, 10% FBS, and 50 μg/mL penicillin containing RPMI 1640 culture (WelGene, Daegu, Korea), and liver cancer cell line (SK-HEP-1, Korean Cell Bank, Seoul, Korea) was cultivated in DMEM culture (WelGene, Daegu, Korea) under the same conditions. Fibroblasts have been used for the separation of the foreskin during phimosiectomy. Following the skin biopsy method by Rheinward and Green (Rheinwald J G, Green H. Serial cultivation of strains of human epidermal keratinocytes; the formation of keratinizing colonies from single cells. Cell 1975;6:331-43.), the separated tumor cells were cultivated in a 10% fetal bovine serum (FBS), 50 μg/mL of streptomyocin, and 50 μg/mL of penicillin containing DMEM culture.

(Toxicity Experiment of Various Cell Types)

The cells cultivated in the medium were divided into 24 wells (4×10⁴/well), and were then cultivated in a medium without FBS for 24 hours. Cytotoxic effect was measured using MTT (3,4-dimethylthiazol-2-yl)2,5-diphenyltetrazolium bromide) assay (Kim, D. S., Jeon, S. E., Park, K. C. Cell Signal, 16, 81-8, 2004).

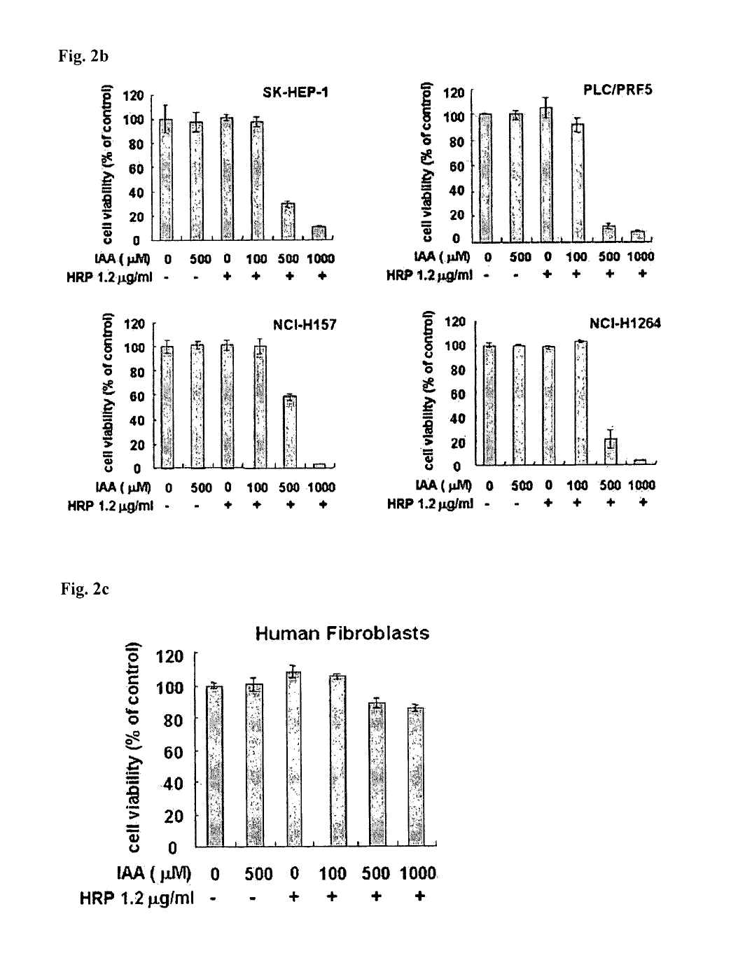

An HRP (1.2 mg/ml) treated group and a group untreated with HRP was separated, each with a final IAA concentration between 1 and 1000 mM in the culture. After cultivating for 20 hours, 0.5 mg/Ml of MTT was added to the culture and cultivated for 4 more hours. After adding and dissolving 1 ml of dimethylsufoxide solution in the well, the absorption was measured using ELISA reader at 540 nm. The results for absorption and cell viability rate were calculated and are shown in FIG. 2a through 2c. The viability rate for the control group in FIG. 2a through 2c was set at 100%.

(Calculations)
Cell viability(%)=(experimental group/control group absorption)×100

The results shown in FIG. 2a through 2c show that administration of IAA alone was not toxic to either normal cells or cancer cells, however, IAA administered with HRP was toxic to most of the cancer cells but not toxic to normal human fibroblasts.

### EXAMPLE 3

### Cytotoxic Effects of IAA with UVB Irradiation

After culturing various types of cells, IAA/UVB treatment was administered. Cell viability was measured by MTT assay. Results showed that IAA/UVB was toxic to cancer cells but normal human fibroblasts were resistant to IAA/UVB treatment.

(Cell Culture) After cultivating G361 human melanoma cell line (ATCC, Rockville, Md.) in a 5% CO₂, 37□, 10% fetal bovine serum (FBS), 50 μg/mL streptomyocin, and 50 μg/mL penicillin containing DMEM culture, mouse melanoma cell line B16 (Korea Cell Bank, Seoul, Korea) liver cancer cell (SK-HEP-1, Korea Cell Bank, Seoul, Korea) was cultivated in the DMEM culture under the same conditions. Fibroblasts have been used for the separation of the foreskin during phimosiectomy. Following the skin biopsy method of Rheinward and Green (Rheinwald J G, Green H. Serial cultivation of strains of human epidermal keratinocytes; the formation of keratinizing colonies from single cells. Cell 1975;6: 331-43.), the separated tumor cells were cultivated in a 10% fetal bovine serum (FBS), 50 μg/mL of streptomyocin, and 50 μg/mL of penicillin containing DMEM culture.

(Toxicity Experiment of Various Cell Types)

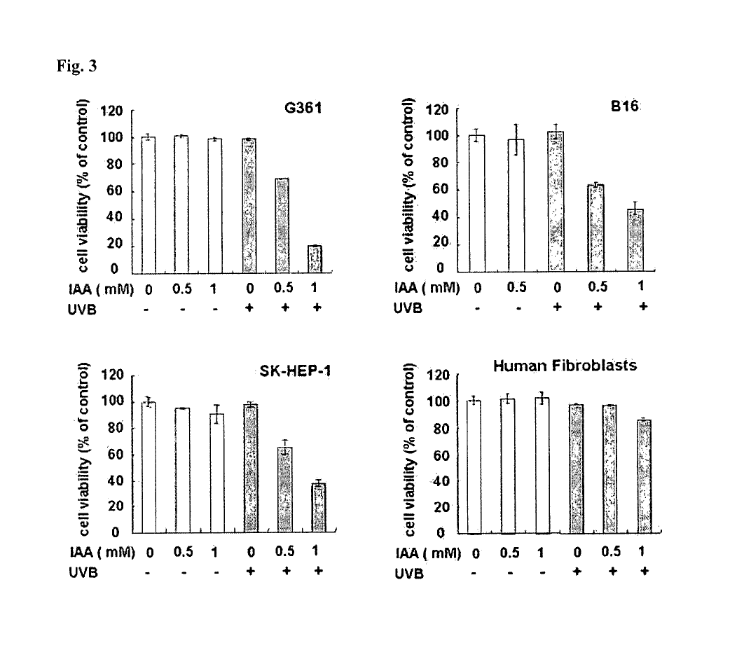

The cells cultivated in the medium were divided into 24 wells (4×10⁴/well), and was then cultivated in a medium without FBS for 24 hours. Cytotoxic effect was measured using MTT (3,4-dimethylthiazol-2-yl)2,5-diphenyltetrazolium bromide) assay. An ICA group treated with UVB (100 mJ/cm²) and a group with ICA alone were separated, each with a final IAA concentration between 1 and 1000 mM in the culture. After cultivating for 20 hours, 0.5 mg/Ml of MTT was added to the culture and cultivated for 4 more hours. After adding and dissolving 1 ml of dimethylsufoxide solution in the well, the absorption was measured using ELISA reader at 540 nm. The results for absorption and cell viability rate were calculated and are shown in FIG. 3. The viability rate for the control group in FIG. 3 was set at 100%.

(Calculations)
Cell viability(%)=(experimental group/control group absorption)×100

The results shown in FIG. 3 show that administration of IAA alone was not toxic to either normal cells or cancer cells, however, IAA treated with UVB was toxic to most of the cancer cells but not toxic to normal human fibroblasts.

### EXAMPLE 4

### Photo-Activation of IAA by Different Wavelength of Light

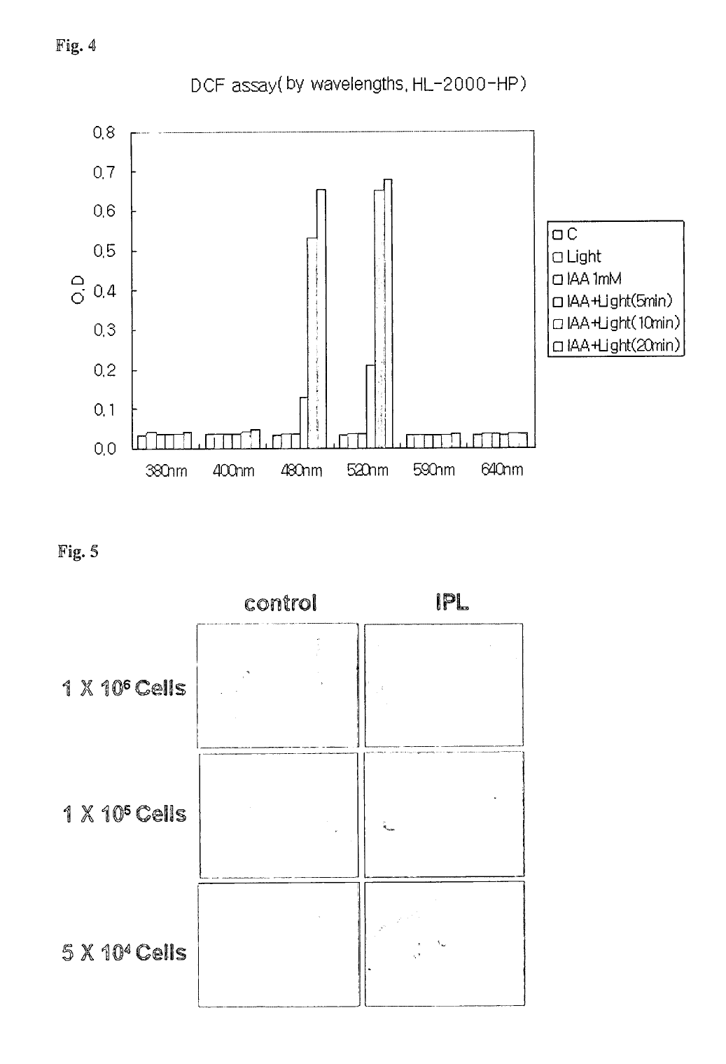

In order to study the effects of different wavelength of light, IAA was irradiated with HL-2000-HP (Ocean Optics, Dunedin, Fla., USA). Free radical formation was measured by DCF (dichlorofluorescein) assay. In order to induce activity of DCFH-DA, DCFH-DA was dissolved to a 1 mM concentration in 100% ethanol. 350 ul of the solution was then added to 1.75 ml of 0.01 N NaOH and allowed reaction for 5, 10, and 20 minutes. The activated DCFH-DA solution was then prepared by mixing 17.9 ml of 25 mM natrium-phosophoric acid buffer solution (pH 7.2). 1 mM of IAA was then administered to the activated DCFH-DA, and using HL-2000HP light irradiator, various wavelength filters (380, 400, 480, 520, 590, 640 nm) (Thorlabs, Inc., Long Beach, Calif., USA) were used to observe any photoactivation of IAA using each wavelength. Results are shown in FIG. 4. Absorption was measured using ELISA reader at 490 nm. As shown in FIGS. 5 through 20, 480 nm (blue) and 520 (green) wavelengths were particularly effective in the activation of IAA.

### EXAMPLE 5

### Treatment of Cancer with IPL Alone

In consideration of the difficulty to rely on the clinical effectiveness of the diode light source, IPL (Intense Pulsed Light) was used for irradiation of particularly strong energy light in animal experimentation of the present invention. In order to test the effectiveness of cancer treatment with IPL alone, the IPL was injected into tumor cell and examined after 1 day. After conferring with histology experts (the results of IPL after 4 days are shown in FIG. 5), it was confirmed that there was no evidence of cell necrosis within the tumor which implies that there was no cytotoxic effect of IPL alone.

(Cancer Cell Transplantation)

Human lung cancer cell NCI-H1246 cells were washed in a 0.1M PBS solution (pH 7.2) and converted into cell samples (1×10⁶, 1×10⁵, and 5×10⁴ ) and intravenously injected using a 30 G syringe into nude mouse (Charles River Lab. Wilmington, Mass., male, 6 weeks old, weight 22-25 g). Evidence of tumor formation was observed after 4 days.

(IPL Examination)

Intense pulse light (IPL) flash lamp by Eclipse was used for visible light irradiation. 20 Irradiation of 20 J/cm² intensity was used with a IPL apparatus able to irradiate between 515 nm to 1200 nm. After applying transmission gel (PROGEL DA-YO Medical, Seoul, Korea) to areas of tumor formation each region was irradiated twice using IPL.

### EXAMPLE 6

### Treatment of Cancer by Combination of IAA and IPL

Human lung cancer cells (NCI-H1264 cell, 1×10⁶) were subcutaneously injected into nude mouse (Charles River Lab. Wilmington, Mass./male, 6 weeks old, weight 22-25 g). It was found that tumor mass was observed 4 days after 1×10⁶ cancer cell injection. 4 and 7 days after cancer cell injection, these mice were injected with IAA (50 mg/kg). Thirty minutes after intravenous injection of IAA, mice were irradiated with IPL (20 J/cm2). Twelve days after cancer cell injection, biopsy was done and tunnel stain was also performed. Results showed that IAA with IPL can induce tumor cell death and is considered to be effective in the treatment of diseases including cancer.

(IAA Administration)

Experimental IAA (10 mg/ml in 50 mM NaHCO/2% v/v ethanol/water, pH 7) was prepared. A control group of mice with tumor mass formation and an experimental group of mice injected with IAA (pH 7, 50 mg/kg) were separated. The injected group was again separated into two groups, one group treated with IAA after 4 days of cancer cell injection and the other after 7 days. Thirty minutes after intravenous injection of IAA, mice were irradiated with IPL (20 J/cm2).

(TUNEL Testing Method)

A TUNEL (Chemicon, Temecula, Calif.) assay kit was used for the present experiment. Simply explained, after the injection of IAA and IPL irradiation, 12 days passed before the nude mouse tissue sample was placed in 10% formalin for 24 hours wherein cell permeability was increased using 0.1% Triton X-100. DNA fragments were labeled by terminal deoxynucleotidyl transferase and anti-digoxigenin peroxidase conjugate. Chemiluminescent peroxidase substrate, diaminobenzidine was then used for observation.

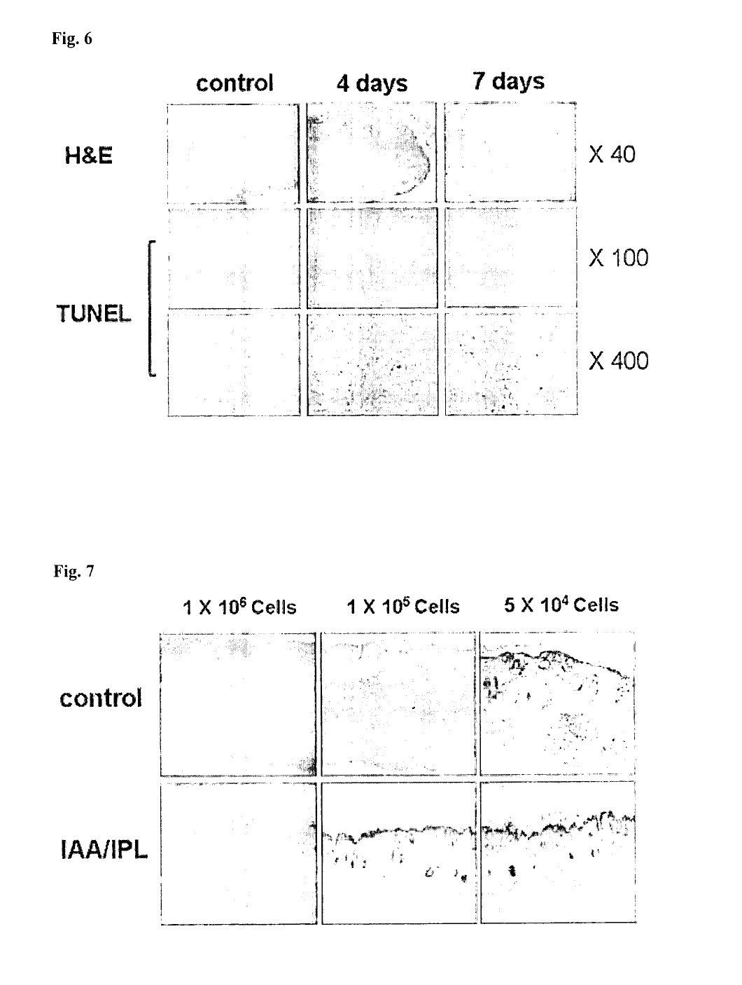

The results of the experiment can be seen in FIG. 6. As shown by the results of the Hematoxylin & Eosin stain and TUNEL assay, IAA and IPL used in parallel can induce tumor necrosis and can be used as an effective means for treating cancer.

### EXAMPLE 7

### Prevention of Cancer by Combining IAA and IPL

It was observed that the combination of IAA and IPL could induce cancer cell necrosis. However, prevention of histologic or distant metastasis is more important in the treatment of cancer. In order to study the prevention effect, IAA and IPL treatment was performed after cancer cell injection but before the appearance of tumor mass. One, three, and five days after NCI-H1264 cell injection, IAA and IPL treatment was performed. Ten days after cancer cell injection, biopsy was done. These results can be seen in FIG. 7. As expected, tumor cell growth was observed in the control tissue but not observed in treated tissues. Thus, it can be said that IAA and IPL treatment was effective in the prevention of distant metastasis.

What is claimed is:

1. A photosensitizer for treatment or prevention of cancer, which comprises a compound of formula (I) or a pharmaceutically acceptable salt thereof: 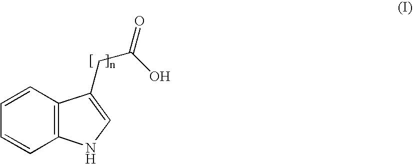
wherein n is an integer of 0 to 3.

2. A pharmaceutical composition which comprises a compound of formula (I) or a pharmaceutically acceptable salt thereof, and a pharmaceutically acceptable carrier or diluent: 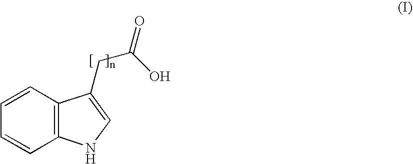
wherein n is an integer of 0 to 3.

3. The pharmaceutical composition of claim 2, wherein the compound of formula (I) is photosensitized by light of wavelength of 280 nm to 1,000 nm.

4. The pharmaceutical composition of claim 2, wherein the compound of formula (I) is contained at a concentration of 0.001 wt % to 30 wt % for photodynamic therapy.

5. The pharmaceutical composition of claim 4, wherein the compound of formula (I) is photosensitized by light of wavelength of 280 nm to 1,000 nm.

6. The pharmaceutical composition of claim 4, wherein the compound of formula (I) is photosensitized by light of wavelength of 350 nm to 450 nm, light of wavelength of 400 nm to 500 nm, or light of wavelength of 500 nm to 600 nm.

7. The pharmaceutical composition of claim 4, wherein the formulation of said composition is in the form of one selected from the group consisting of liquid, semisolid, solid and aerosol.

8. The pharmaceutical composition of claim 7, wherein the formulation of said composition is in the form of one selected from the group consisting of aqueous or non-aqueous suspension, solution, cream, ointment, gel, syrup, suppository, tablet, capsule and micro-droplet spray.

9. The pharmaceutical composition of claim 4, wherein the photodynamic therapy is applied to the treatment or prevention of skin or skin related diseases, oral and gastrointestinal tract diseases, urology or urology related diseases, respiratory or related diseases, circulatory or related diseases, diseases related to head and neck, lymphoreticular disorders, and infectious disease including miro-organism, virus and parasitic disorders.

10. A method for administrating the pharmaceutical composition according to claim 2, wherein the administering route is selected from the group consisting of topical application, intravenous injection, intramuscular injection, intra-cranial injection, intra-tumoral injection, intraepithelial injection, trans-epidermal injection, esophageal administration, intra-peritoneal administration, intra-arterial injection, intra-articular injection and oral administration.

11. A photodynamic therapy kit which comprises:
i) a pharmaceutical composition containing a compound of formula (I) at a concentration of 0.001 wt % to 30 wt %; and
ii) a light emitting device for irradiation of light of wavelength of 280 nm to 1,000 nm.

12. The photodynamic therapy kit of claim 11, wherein the light emitting device irradiates ultraviolet rays of wavelength of 350 nm to 450 nm, blue light of wavelength of 400 nm 500 nm, or green light of wavelength of 500 nm 600 nm.

13. The photodynamic therapy kit of claim 11, wherein the light emitting device is one selected from the group consisting of light emitting diode, laser diode, dye laser, halogen metal lamp, flash lamp, filtered fluorescent or any kinds of lamp for photodynamic therapy, and any system for the delivery of light to inside of the body through laser fiber.

14. The photodynamic therapy kit of claim 11, wherein the intensity of light irradiated by the light emitting device is 1 J/cm² to 100 J/cm².

15. The photodynamic therapy kit of claim 14, wherein the pulse duration time of light irradiated by the light emitting device is between 0.1 ms and 500 ms, and the number of irradiation is between 1 and 100.

16. The photodynamic therapy kit of claim 11, said kit being used for the treatment or prevention of one selected from the group consisting of skin or skin related diseases, oral and gastrointestinal tract diseases, urology or urology related diseases, respiratory or related diseases, circulatory or related diseases, diseases related to head and neck, lymphoreticular disorders, and infectious disease including
